(19) United States
(12) Patent Application Publication
(10) Pub. No.: US 2016/0231575 A1
(43) Pub. Date: Aug. 11, 2016

(54) SPATIOTEMPORAL FOCUSING APPARATUS AND METHOD

(71) Applicant: Technion Research & Development Foundation Limited, Haifa (IL)

(72) Inventors: Shy SHOHAM, Haifa (IL); Tom MAYBLUM, Tel-Aviv (IL); Adi SCHEJTER BAR-NOAM, Haifa (IL)

(21) Appl. No.: 15/017,999

(22) Filed: Feb. 8, 2016

Related U.S. Application Data
(60) Provisional application No. 62/113,459, filed on Feb. 8, 2015.

(51) Int. Cl. G02B 27/09; H01S 3/00; G02B 5/18; G02B 27/42; G02B 26/10; A61N 5/06; G02B 21/00; G02B 27/30; G02B 5/04; G02B 27/10; G02B 27/16; G02B 27/22; G03H 1/00

(52) U.S. Cl. CPC G02B 27/0905; G02B 27/2292; H01S 3/0057; G02B 5/1814; G02B 27/4277; G02B 27/4294; G02B 26/105; G03H 1/0005; G02B 21/0048; G02B 27/30; G02B 27/0944; G02B 27/0927; G02B 5/04; G02B 21/006; G02B 21/0064; G02B 27/1086; G02B 27/16; A61N 5/0622; G03H 2001/005; G03H 2001/0094; G03H 2001/0077; A61N 2005/067

(57) ABSTRACT

Optical apparatus comprises an impulse producing laser followed in an optical path by a spatio-temporal focusing element which provides spectrally dispersed and typically collimated beams. The spatio-temporal focusing element is then followed in the optical path by a volumetric projection system which provides a volumetric image from the collimated beams, the volumetric image extending over a plurality of planes in a volume.

### RELATED APPLICATION

This application claims the benefit of priority under 35 USC §119(e) of U.S. Provisional Patent Application No. 62/113,459 filed on Feb. 8, 2015, the contents of which are incorporated herein by reference in their entirety.

### FIELD AND BACKGROUND OF THE INVENTION

The present invention, in some embodiments thereof, relates to a spatiotemporal focusing apparatus and method and, more particularly, but not exclusively, to such a spatiotemporal focusing apparatus and method that is able to focus in multiple planes.

Two-photon laser scanning microscopy is a widely used tool for optical imaging, mainly due to its excellent optical sectioning capabilities, even deep inside scattering tissues. However, the point-by-point scanning process is often considered the limiting bottleneck for various applications such as rapid functional imaging and stimulation of neural activity, even in the context of rapid scanning strategies.

U.S. Pat. No. 7,698,000 discloses an optical technique known as temporal focusing. A temporal pulse manipulator is configured to affect trajectories of light components of an input pulse impinging thereon so as to direct the light components towards an optical axis of a lens along different optical paths. The temporal pulse manipulator unit is accommodated in a front focal plane of the lens, thereby enabling to restore the input pulse profile at the imaging plane.

Temporal focusing allows to simultaneously illuminate a single line or a plane inside a volume of interest while maintaining optical sectioning. Temporal focusing (TF) multiphoton excitation provides an alternative approach that facilitates extended area illumination with tight optical sectioning by decoupling the axial and lateral resolutions. This decoupling is achieved by separating the laser pulse into beamlets that propagate in different directions until they coincide in the focal plane, causing the pulse duration to reach a minimum at the focal plane, and to be longer in any adjacent plane. Since the two-photon fluorescence signal is inversely proportional to the pulse width, axial sectioning is achieved, regardless of the spatial focusing. Recently, TF systems have demonstrated superior capabilities in the fields of functional imaging and photo-stimulation, as well as other applications. Despite these major advantages, conventional TF designs have an important drawback, and that is that they are limited to excitation in a single plane. Referring now to FIG. 1, in these systems the general design is as follows: a laser beam 10 from a rapid pulsing laser arrives at laser directing unit 12, for example a Spatial Light Modulator (SLM), or alternatively galvanometric mirrors, or a cylindrical lens. The directing unit 12 guides the light towards a temporal focus (TF) element 14 such as a grating or a diffuser, and onto a biological sample 16 using lenses such as objective lens 18.

In parallel to these developments, work done in the femtosecond laser micromachining community has explored the use of spatio-temporal focusing for shaping the cross section of the focal spot using a grating-pair optical design and then allowing both spatial and temporal focusing to simultaneously occur in a single focal spot. The solution is limited to illumination of a single spot in the center of a single optical focal plane.

These current solutions are not only highly customized but they limit the excitation to a single plane, onto which TF focuses the illumination. US Patent Application 2014/0313315, Dana & Shoham, Method and System for Transmitting Light), discloses a temporal focusing system, which is configured for receiving a light beam pulse and for controlling a temporal profile of the pulse to form an intensity peak at a focal plane. The temporal focusing system has a prismatic optical element to receive the light beam pulse from an input direction parallel to or collinear with the optical axis of the temporal focusing system and which diffracts the light beam pulse along the input direction. systems. There are thus disclosed methods that allow to easily switch a TF module into and out of an optical system, thus providing hybrid operation, which includes scanning a single temporal focus plane in a 3D volume. Although such a solution does allow volumetric scanning, there is only one focal plane at any given time. To address this challenge US Patent Application 2014/0313315 also discloses a multi-element replicator, however the replicator requires a large number of components, and is highly limited since the resultant light distribution is not simply axially shifted, and also contains undesired lateral shift.

Over the last few years, optogenetics has emerged as the central strategy for optically controlling neural activity using light-sensitive ion channels or pumps for stimulation or inhibition. It is widely accepted that cell-targeted stimulation in 3-dimensional, scattering tissue is highly challenging and can currently only be achieved using multiphoton optogenetic stimulation. However, multiphoton optogenetics poses several fundamental challenges, in particular, in the attempt to achieve effective multiphoton excitation of large membrane patches while maintaining adequate optical-sectioning. FIGS. 2A-2C provide an illustration of common multiphoton optogenetics illumination strategies. FIG. 2A shows a typical neuron 20 illuminated by a diffraction limited spot 22 and it is immediately clear that spot 22 is unable to excite much of the membrane of the cell 20. As shown in FIG. 2B, using a lower NA objective lens may enlarge the spot laterally but will deteriorate the optical sectioning significantly and thus will not provide single neuron resolution. Temporal focusing (TF) as shown in FIG. 2C provides a larger spot 26. TF decouples the lateral and axial focal dimensions by varying the pulse duration along the propagation direction, thereby allowing scan-less illumination of relatively large light spots.

Indeed, in addition to its multiple applications in microscopy and micromachining, TF has also been used to realize photo-stimulation systems with extended membrane coverage. Multi-cell stimulation in these studies has been obtained using either conventional galvanometer scanners for sequential scanning, or holographic patterning for simultaneous scanning. However, both types of TF systems constrain excitation to a specific 2D plane and are relatively complex to realize and to integrate into existing microscopes. Moreover, the holographic solutions also suffer from significant holographic speckle which is high frequency noise. Holographic strategies that avoid the speckle problem, such as hybrid solutions with mechanical scanning or time-averaging of multiple hologram projections suffer from low temporal accuracy, while speckle-free alternatives like Generalized Phase Contrast offer low efficiency for sparse large-field stimulation and also limit excitation to 2D.

### SUMMARY OF THE INVENTION

As mentioned above, in parallel to these developments, work done in the femtosecond laser micromachining community explored the use of spatio-temporal focusing for shaping the cross section of the focal spot. Interestingly, the basic grating-pair optical design used in these studies is crucially different from the single grating design used in microscopy and optogenetics TF systems, in that the second grating recollimates the beam, leading to a spectrally dispersed collimated beams that upon being focused allows both spatial and temporal focusing to simultaneously occur in a single focal spot, in what is referred to herein as simultaneous spatial and temporal focusing or SSTF. The micromachining community always used to illuminate a single spot in the center of the optical focal plane, however as the beam is recollimated, the present embodiments provide the beam to a directing unit or replicator or both to provide multiple axially shifted optical focal planes with illuminated spots and allow the spots to be scanned within their respective optical focal planes, thus providing spatio-temporal focusing in a three-dimensional volume. That is to say, the order of beam director and temporal focusing unit may be reversed.

According to an aspect of some embodiments of the present invention there is provided an optical apparatus comprising an impulse producing laser followed in an optical path by a spatio-temporal focusing element configured to provide spectrally dispersed collimated beams, the spatio-temporal focusing element followed in said optical path by a volumetric projection system configured to provide a volumetric image from said collimated beams, the volumetric image extending over a plurality of planes.

The term spectrally dispersed collimated beams refers to the way in which the spatio-temporal focusing element may split the collimated multispectral beam from the impulse producing laser into separate spectra and then collimate the separate spectra.

In an embodiment, said planes are excitation planes for excitation of fluorescent materials or optogenetic probes.

In an embodiment, said impulse producing laser is a femtosecond laser.

In an embodiment, said temporal focusing element comprises two gratings or two dual prism gratings (DPG).

An embodiment may comprise an adjustment mechanism for adjusting a distance between said two DPGs, thereby to adjust a focal spot.

An embodiment may comprise a scanner in said optical path, the scanner comprising a plurality of galvanometric mirrors to scan each spot over a respective replication plane.

An embodiment may comprise a holographic projection element in said optical path, the holographic projection element configured to provide multiple projections of each spot over a respective focal plane simultaneously.

An embodiment may comprise a scanner comprising a plurality of galvanometric mirrors to scan each spot over a respective focal plane, and further comprising a holographic projection element configured to provide multiple projections of each spot over a respective focal plane simultaneously.

In an embodiment, said temporal focusing unit comprises two prisms and two gratings.

In an embodiment, said temporal focusing unit comprises a plurality of mirrors and at least two gratings, configured to provide collimated beams for different wavelengths of said impulse.

In an embodiment, said temporal focusing unit is modified to elongate said spot into a line.

In an embodiment, said temporal focusing unit is characterized by an optical axis and is configured for receiving said impulse and for controlling a temporal profile of said impulse to form an intensity peak at said focal plane, said temporal focusing unit having at least two prismatic optical elements configured for receiving said light beam pulse from an input direction parallel to or collinear with said optical axis and diffracting said light beam pulse along said input direction.

In an embodiment, a replicator provides multiple axially shifted optical focal planes. The replicator may comprise a plurality of beam splitters and mirrors to provide a plurality of optical paths of different lengths.

The embodiments may be used for optogenetic control of a single cell or for optogenetic control of a plurality of cells in three dimensions, or for micromachining or microscopic imaging or photo-manipulation or micro-printing. In the case of micro-printing, the focal planes include a photo-activatable material.

According to a second aspect of the present invention there is provided an optical apparatus comprising a temporal focusing unit having two diffraction gratings, a first diffraction grating to separate a laser beam into different wavelengths and a second of said diffraction gratings to recollimate said separated beam, said unit being followed by a replicator configured to provide a plurality of optical paths for said recollimated beam, thereby to provide a plurality of focal planes.

According to a third aspect of the present invention there is provided a method of providing three-dimensional focusing comprising:

providing a femtosecond pulse beam;

providing a first grating to split said femtosecond pulse into a plurality of wavelengths;

providing a second grating to recollimate said beam;

splitting said beam into a plurality of optical paths, thereby to provide a plurality of focal planes for said beam.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified schematic diagram showing a currently practiced arrangement for temporal focusing, which achieves a single focal plane;

FIGS. 2A, 2B and 2C are simplified schematic diagrams showing three different schemes for focusing according to the current art;

FIG. 3 is a simplified schematic diagram showing focusing on a neuron according to an embodiment of the present invention;

FIG. 4 is a simplified schematic diagram showing an arrangement for spatial temporal focusing that allows focusing in three dimensions, according to an embodiment of the present invention;

FIG. 5 is a simplified schematic flow diagram showing operation of an optical method according to an embodiment of the present invention;

FIGS. 6A and 6B are simplified schematic diagrams showing focusing serially and simultaneously on different cells in different planes according to two different embodiments of the present invention;

FIG. 7 is a simplified schematic diagram showing a replicator according to an embodiment of the present invention;

FIGS. 8A, 8B and 8C are three simplified schematic diagrams showing views of a spot, graphs of the spot size and stability of the spot size across a field of view according to an embodiment of the present invention;

FIGS. 9A, 9B and 9C are three simplified schematic diagrams showing results in terms of spots in two and three dimensions according to an embodiment of the present invention; and

FIG. 10 is a simplified schematic diagram illustrating an experimental apparatus according to an embodiment of the present invention.

### DESCRIPTION OF SPECIFIC EMBODIMENTS OF THE INVENTION

The present invention, in some embodiments thereof, relates to a spatiotemporal focusing apparatus and method and, more particularly, but not exclusively, to such a spatiotemporal focusing apparatus and method that is able to focus in multiple planes.

Temporal focusing (TF) multiphoton systems constitute a solution for a range of high-resolution photo-manipulation and imaging applications requiring axially sectioned excitation. The present embodiments may provide a solution for obtaining TF in a flexible three-dimensional pattern rather than in a single TF plane as heretofore known. The present solution employs a spatio-temporal focusing (SSTF) add-on module that can be integrated in front of essentially any multiphoton imaging or stimulation system. Optionally, a 3D multi-focal module can be integrated into the system to achieve multi-plane replication.

The present embodiments disclose a solution based on two optional subunits which can potentially be combined, that can be easily integrated into multiphoton microscopes for focusing TF light to multiple planes simultaneously in a 3D volume. The first subunit may turn the optical system into an SSTF system wherein the focal shapes are naturally focused within the entire 3D volume and not just in a select 2D plane, thus providing flexible 3D patterns. A pre-dispersion unit, which may be inserted into the original light-path, creates an SSTF collimated light beam that reaches a directing unit, made up for example of SLM or galvo mirrors, which guides the light into the microscope. In the second subunit, which may be placed inside the microscope, a temporally-focused excitation plane is replicated onto multiple axially-displaced planes within a volume.

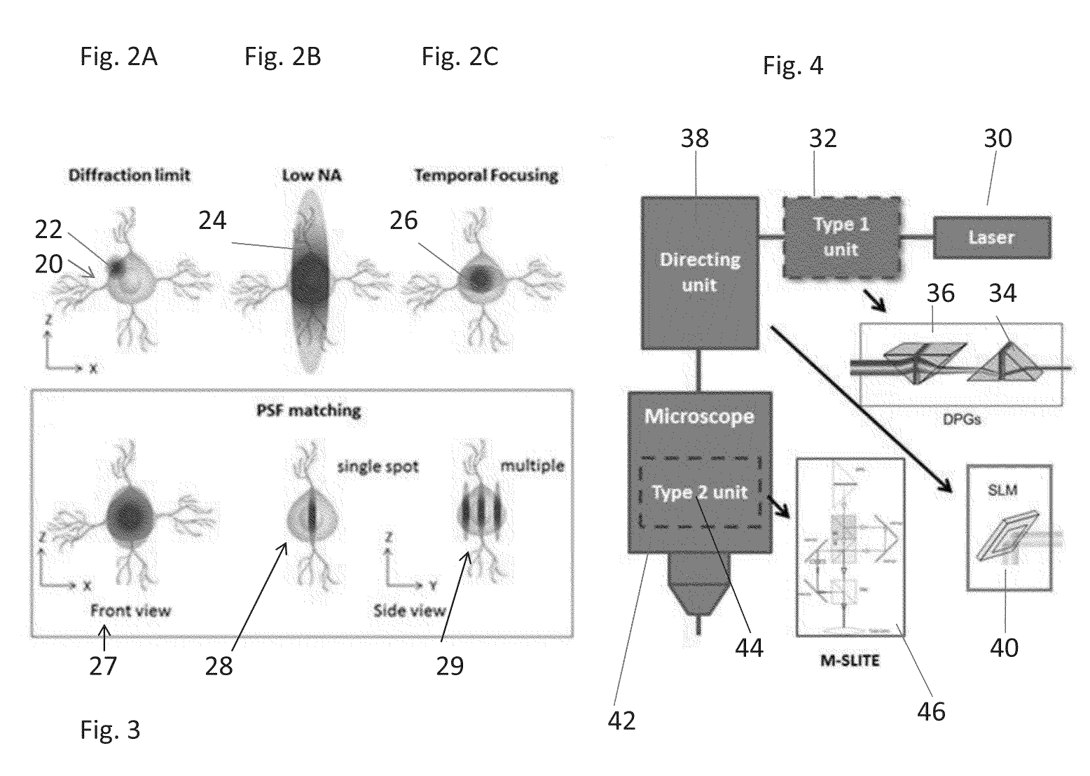

The present embodiments thus disclose TF-based solutions for providing illumination patterns in three dimensions. One application of such a system is effective multiphoton optogenetic stimulation, which is the method used for optical cell-targeted stimulation in 3-dimensional tissues such as the brain. A major challenge in such systems is the lack of effective excitation of a single neuronal cell, where the expression of light-gated ion channels is limited to the membrane of the cell. As discussed above in respect of FIG. 2A, a diffraction-limited spot will not excite enough membrane channels and thus requires a scanning mechanism in order to provide efficient excitation, but the downside is worse temporal resolutions of controlled stimulation. As shown in FIG. 2B, using a lower NA objective lens may enlarge the spot laterally but will deteriorate the optical sectioning significantly and thus will not provide single neuron resolution. As shown in FIG. 2C, TF has been suggested and successfully used in multiple realizations to create more efficient excitation, by generating a wide lateral spot with good optical sectioning. However in the prior art the illumination was limited to a single plane, and neurons in vivo are not restricted to a single plane.

The present embodiments may provide a solution for the challenges of multiphoton optogenetics and Holographic Optogenetic Neural Stimulation (HONS), which builds upon and extends the micromachining grating-pair SSTF approach. In the present embodiments, SSTF is used to shape a disc-like focal spot that approximately fits the size of a target nerve cell in both the lateral and axial dimensions. The present embodiments may be seamlessly integrated into the optical path before essentially any multiphoton imaging or stimulation system. The embodiments may thus allows easy switching between imaging and cell-targeted stimulation, as well as simultaneous holographic multi-spot stimulation, while avoiding the effect of holographic speckle, since each cell can be efficiently covered by a few sparse spots.

After describing the optical design, the generation of a spatially invariant disc-shaped focal spot which approximately matches cellular dimensions is discussed. The configuration enables random access illumination by mechanically scanning over preselected single cells. The same SSTF configuration is then shown in combination with holographic patterning, in order to experimentally demonstrate the simultaneous generation of multiple disc-shaped spots distributed in 3D.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

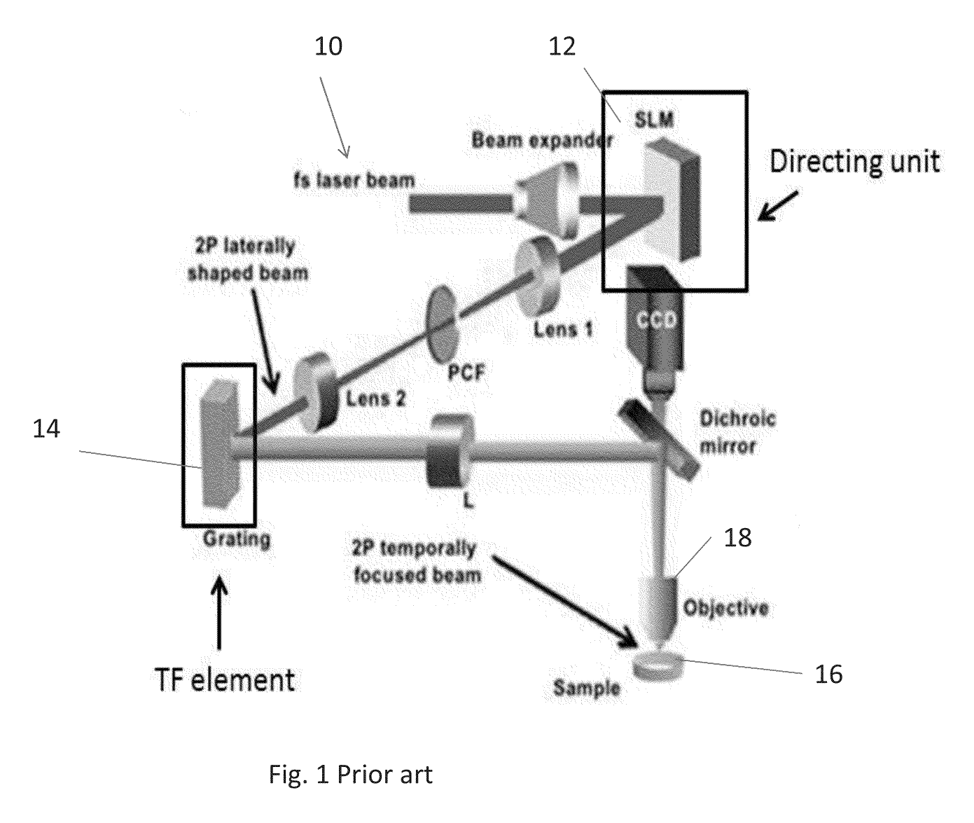

Reference is now made to the drawings. FIGS. 1 and 2A-2C represent the prior art and are discussed above.

FIG. 3 illustrates illumination geometries that may be provided using the present embodiments and a spatio-temporally focused spot. Numeral 27 shows a front view in which the spot extends over the neuron. The middle view—28—shows the same spot from the side and it is clear that the illuminated plane lacks depth. The right hand view—29—shows three different planes being simultaneously illuminated within the volume of the neuron.

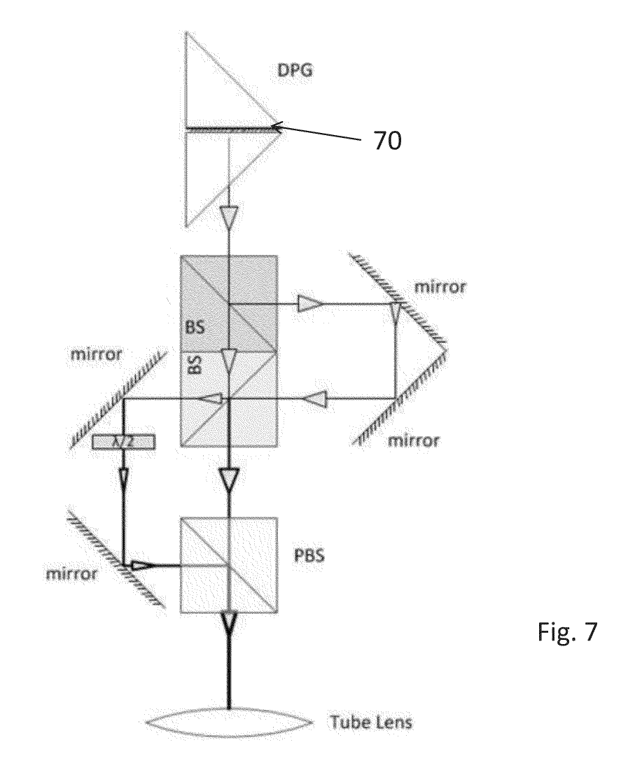

Reference is now made to FIG. 4, which is a simplified diagram illustrating a schematic of a system according to the present embodiments. As shown there is provided a laser 30, a Type 1 unit 32, introduced before the microscope system, which spectrally disperses the beam into separate spectra and then collimates the resulting spectral beams to produce parallel beams of different spectra. The unit may be made up for example, of two DPGs 34 and 36. A directing unit 38 may comprise scan mirrors or an SLM 40 for scanning or simultaneous projection, or a hybrid of both, and thus volumetric projection. The directing unit is followed by microscope 42. A replicator unit, here shown as the Type 2 unit 44, can optionally be integrated into the microscope 42. The replicator unit may for example be an M-SLITE 46, discussed in greater detail with respect to FIG. 7 below. The optical apparatus may use a very rapidly pulsed laser to produce what are effectively impulses. Impulses are extremely short duration pulses of large amplitude, and as shown in Fourier analysis produce a very wide bandwidth, even though the laser itself is typically monochromatic. In the present embodiments femtosecond pulses are used to provide good enough approximations of the mathematical impulse and uses of the term ‘impulse’ herein are to be construed accordingly. The laser 30 is followed in the optical path by the spatio-temporal focusing element 32 which provides the parallel and collimated beams as discussed. The temporal focusing element 32 may be followed by a volumetric projection system and optionally by the replicator 44 which provides replications of the spot temporally focused in replication planes axially displaced from each other. As a result, focusing of the light is provided in multiple planes of the 3D volume, thus allowing the situation shown in FIG. 3. In the case of optogenetic stimulation, the planes are excitation planes for excitation of the fluorescent materials.

As shown in FIG. 3, the temporal focusing element 32 comprises two dual prism gratings (DPG) 34 and 36, the first of which may spread out the different wavelengths in the impulse and the second of which may collimate the separated beam. However the combined prism and diffraction grating is only an example and the diffraction gratings and prisms may be provided as discrete elements, and the order between prism and diffraction grating may be varied, as will be discussed below. For example the temporal focusing unit may comprise two prisms and two gratings. Alternatively, the temporal focusing unit may comprise multiple mirrors and at least two gratings, configured to provide collimated beams for different wavelengths of the impulse.

The distance between the two DPGs may be adjusted, and such adjustment provides a way for moving a focal spot.

As mentioned, there are a number of ways of designing directing unit 38. One is a scanner made up of galvanometric mirrors which scan over a respective replication plane. An alternative is a holographic projection element which provides multiple projections over the respective replication plane simultaneously.

A hybrid system may include both the galvanometric mirrors and the holographic projection element.

The temporal focusing unit may be modified to elongate the spot into a line, which can then be scanned over a plane.

The temporal focusing unit may thus be characterized by an optical axis. The impulse is received from the laser and the temporal focusing unit controls a temporal profile of the impulse to form an intensity peak at a focal plane. The temporal focusing unit has the two prismatic optical elements as discussed which receive the incoming light beam pulse from an input direction parallel to or collinear with the optical axis. The first optical element then diffracts the light beam pulse along the input direction into separate beams and the second element collimates the beams.

The replicator 44 may comprise beam splitters and mirrors to provide optical paths of different lengths to provide different focal planes.

As well as optogenetics mentioned above, which may involve illumination or control of a single cell or of multiple cells in three dimensions, other applications include micromachining, microscopic imaging, photo-manipulation and micro-printing.

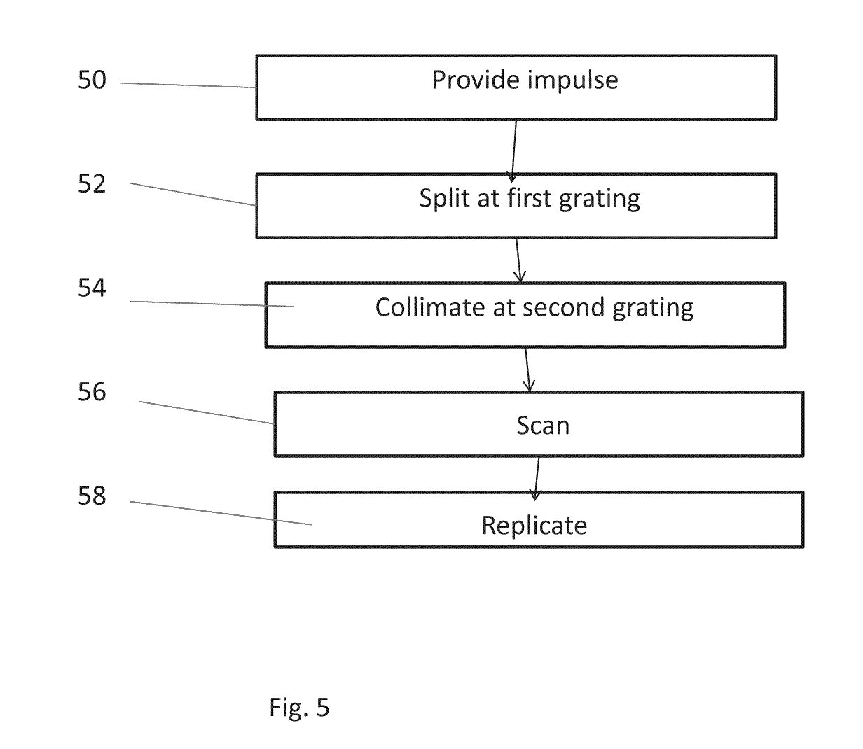

Reference is now made to FIG. 5, which illustrates a method of providing three-dimensional focusing. The method comprises providing 50 an impulse, say a femtosecond laser pulsed beam, providing 52 a first grating to split the femtosecond pulse into constituent spectra, providing 54 a second grating to recollimate the resulting spectral beams, and then splitting 58 the beams into a plurality of optical paths, thereby to make several replications of the focal plane for the beam. In addition the planes may be mechanically or holographically scanned 56 within the volume, that is to say provide volumetric projection, which is typically carried out prior to replication.

The present embodiments thus provide a TF-based pattern illumination system which can be seamlessly integrated into an already existing optical system. In the field of multiphoton optogenetics, the present embodiments may shape the focal spot so that it efficiently matches the dimensions of a common neuron, but in a manner that can directly be applied towards 3D excitation as discussed with respect to FIG. 3. In one realization, the introduction of a module with two dual-prism gratings, known as DPGs or grisms, may provide the SSTF effect on-axis. The effect may be incorporated into an existing microscope setup without any alteration to the microscope itself, since the beams exit the two DPG arrangement on-axis and in collimation. Adjusting the distance between the grating elements provides a simple degree of freedom for adjusting the focal spot, as mentioned above. In particular embodiments, a shaping module may be located in front of a rapid scanning system such as, for example, a galvanometer or AOD scanners, or scanning may use a holographic projection system composed of a phase spatial light modulator. Further embodiments may be a hybrid system combining a holography element and a scan system.

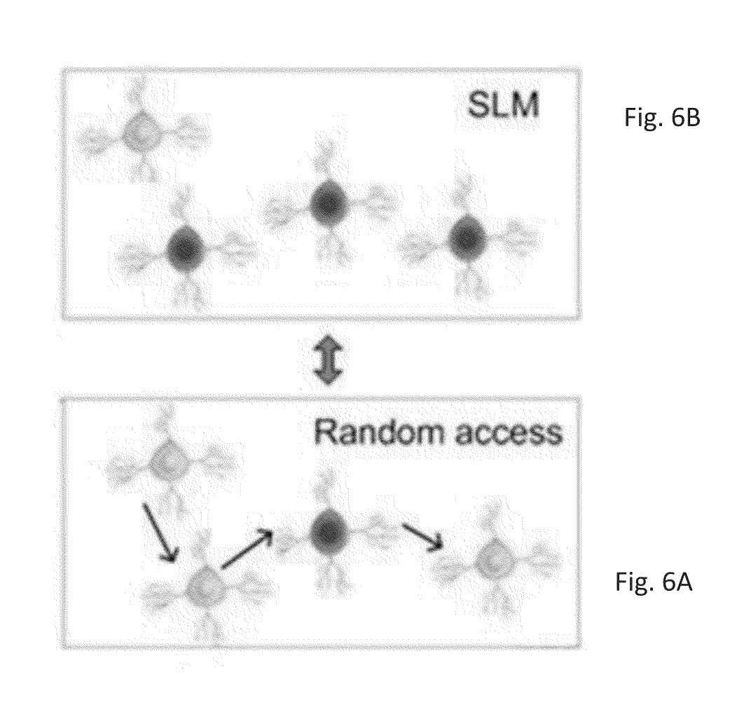

Reference is now made to FIGS. 6A and 6B, which are two figures showing demonstrations of the targeting of cells using disc-shaped spots. The spots are scanned over neurons. In FIG. 6A the focal spot is scanned across the accessible field of view using galvanometric mirrors, so that it is possible to selectively and rapidly target a disc-shaped spot onto different points in the focal plane. As shown in FIG. 6B, the holographic system by contrast can project multiple such spots simultaneously.

In other embodiments, the beam shaping SSTF module may consist of a different arrangement of gratings and prisms—for example a system consisting of only two prisms and two gratings, and not using the two inner prisms illustrated, can be simply designed to provide a similar on-axis solution. Another possibility is a module consisting of several mirrors and two or more gratings, that returns a collimated beam into the optical system input port.

In yet further embodiments the focal spot is not shaped into a disc, but rather into another shape. For example a focal spot can form a short line segment that can be rapidly scanned (in 1D) across the target.

In a further embodiment, the beam shaping module is integrated into the microscope rather than placed in front of the microscope port, and such a construction is possible since the module does not alter the optical axis.

In yet further embodiments, the beam shaping module is not an SSTF module that uses diffractive elements to spectrally disperse the incoming beam, but rather uses another solution known in the art of femtosecond micromachining for shaping the focal excitation spot, for example a cylindrical telescope or an aperture.

In yet other embodiments, the system is used for microscopic imaging, photo-manipulation, printing, or machining using one of the methods known in the art, where the shaped patterned beam is used to provide more rapid access to extended shapes.

Optionally, a multi-plane replication unit can be integrated into the system. Such a multi-plane replication unit, which can optionally be combined with the previous SSTF solution or independently of it, replicates a TF plane to multiple depths by elongating multiple optical paths at various lengths. The design relies on a replicator which is integrated into the microscope. Referring now to FIG. 7, an embodiment is shown termed M-SLITE. In M-SLITE. multiple beams splitters BS and mirrors positioned after a DPG 70 are used to obtain multiple temporally focused excitation planes at the microscope focal plane. The diagram shows three separate beam paths of different length. The M-SLITE forms a replicator, which is the component that provides the different focal planes. The DPG illuminates a line or spot as desired, and then the line or spot is displaced in the focal plane to the multiple planes that the replicator forms. The replicator could also be formed using diffractive elements.

In other realizations, the replicator unit optionally consists of a diffractive element that splits the light into multiple planes simultaneously, as known in the art. In some realizations intensity modulation elements known in the art can optionally be inserted into the light path of FIG. 7, in order to create differential modulation of the different illumination planes, which can be used for example to extract depth information from images that combine multi-depth information.

### EXPERIMENTAL RESULTS

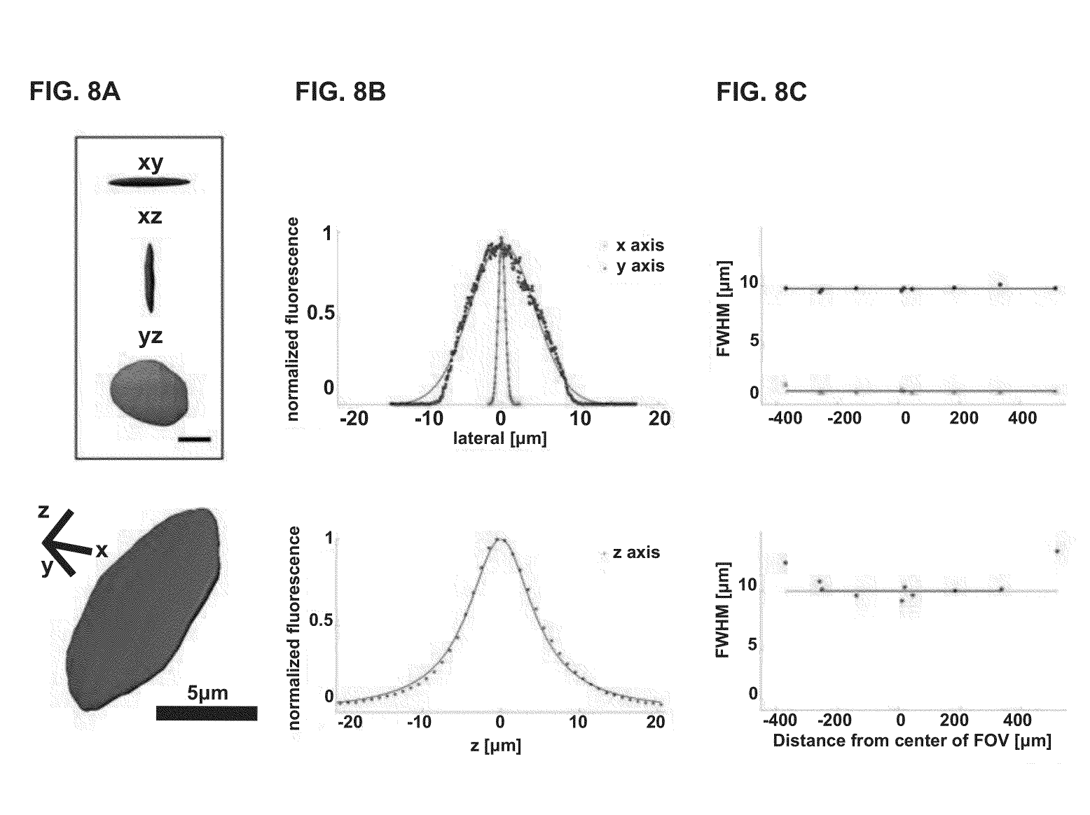

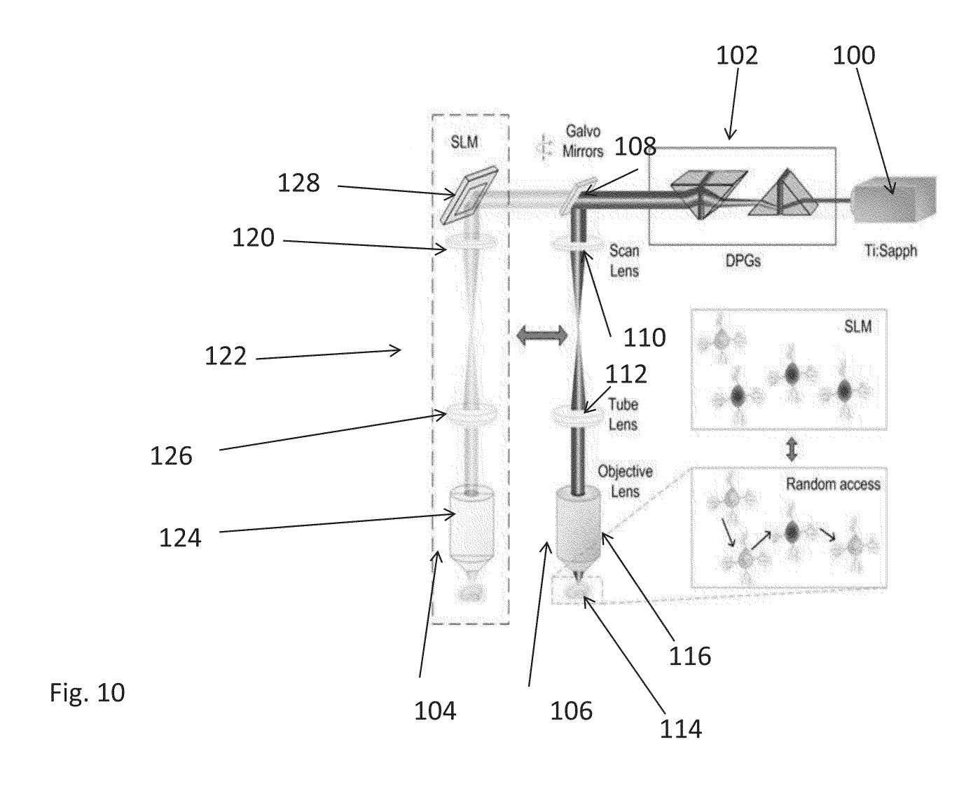

An experimental arrangement was set up using a Ti:Sapphire laser operating at a central wavelength of 920 nm with 70 fs pulse duration and 80 MHz repetition rate. The arrangement was able to demonstrate the generation of the vertical disc-shaped focal spot 80 of FIGS. 8A-C, having a circular cross-section of 10×10 μm and 1 μm width. In order to measure the dimensions of the spot, 1.1 μm fluorescent beads were used and scanned over a volume of 30×30×50 μm. FIG. 8A shows measured dimensions and behavior of the resulting disc-shaped spot and an isometric view of the generated disc-shaped spot. FIG. 8B is a graph showing measured lateral dimensions of the disc, and yielding a line-like shape with a FWHM of 10 μm in the y axis and 1 μm in the x axis, and 10 μm in the z axis. FIG. 8C shows experimental results demonstrating the stability of the disc dimensions across a full field of view. The experimental results are now considered in greater detail with respect to FIG. 10. The experimental setup uses a tunable Ti:Sapphire laser source 100 with a 80 MHz repetition rate and a minimal 70 fs pulse duration to set up what is in effect an impulse. A DeepSee module may be added for pre-chirp compensation. The laser's central wavelength may be configured to match the dual-prism gratings' (DPGs) coating, which may be 920 and 905 nm, for the random access and holographic setups respectively.

For seamless integration of SSTF, a unit of two identical DPGs 102, say at 1,600 or 1,200 lines mm⁻¹, may be positioned at the same orientation one after another, at the entrance to each of two microscope systems 104 and 106. A half-wave plate placed before the polarization-dependent DPGs 102 may be used to achieve maximum intensity at the entrance to the microscope.

For random access, the collimated beam is scanned using galvanometric mirrors 108, relayed through a scan lens 110 and tube lens 112, and then focused onto the sample 114 using a 20× objective 116.

For patterned HONS, the collimated beam is first passed through a scan lens 120, then expanded by a telescope (2:1) 122 in order to fill the phase modulating spatial light modulator. The hologram is relayed onto the back aperture of the objective 124 through a tube lens 126 in order to obtain a desired pattern of light-discs on the sample. An additional half-wave plate is placed before the SLM 128 in order to match its polarization, to achieve maximum intensity in the first order of the hologram.

In order to determine the dimensions of all disc-like spots, 1.1 μm fluorescent beads were scanned and fitted with a Gaussian and a Lorentzian function (for lateral and axial dimensions, respectively) from which the full width at half maximum (FWHM) was derived in each case. Raw images were processed for brightness and contrast enhancement, and analyzed using image processing tools.

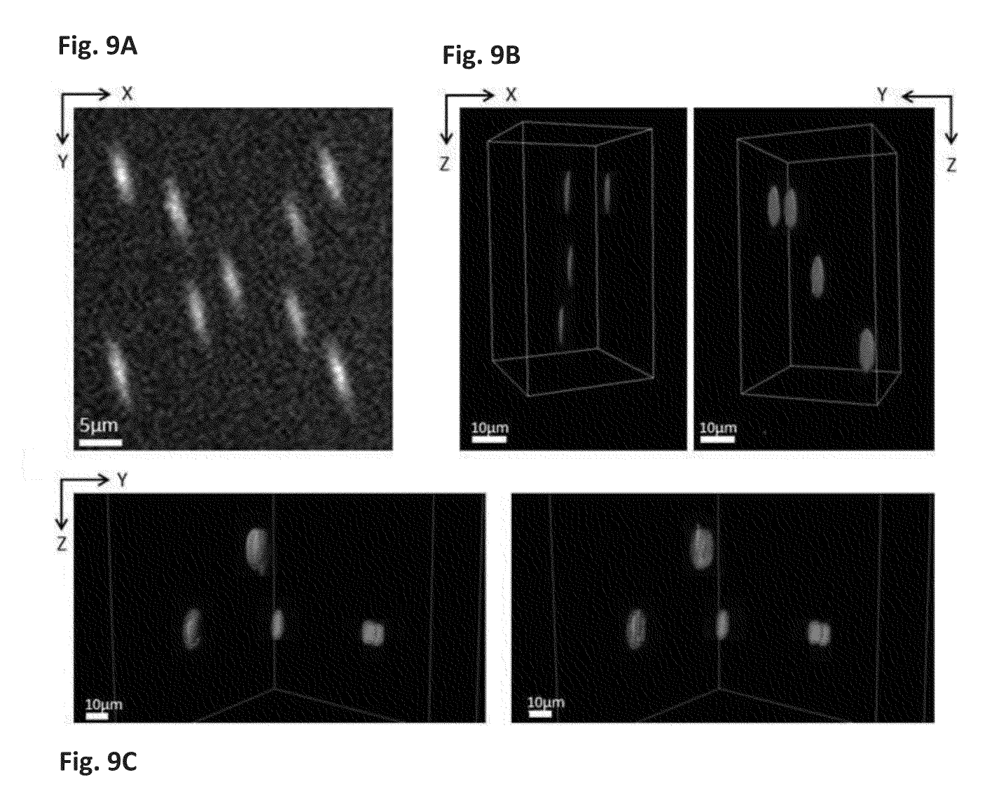

FIGS. 9A-9C show resulting patterns of spots. The light disc generated using the random access configuration is shown in FIG. 9A. The measurements yield a disc shaped spot with a circular vertical cross-section FWHM of 10×10 μm and 1 μm width as shown in FIGS. 9A and 9B. FIG. 9B shows a 3D pattern of light discs from two different views generated using a holographic element. The light-discs are generated simultaneously in three different planes separated by 20 μm each. The system may of FIG. 9B may be used for simultaneous multiple-cell optogenetic control. In order to define the accessible field-of-view (FOV) in which the disc dimensions remain relatively constant, it is possible to measure the dimensions of the generated disc in different lateral locations over the entire FOV, as shown in FIG. 9C. The axial sectioning was maintained over an area of roughly 600×600 μm.

Two- and three-dimensional (2D and 3D) patterns of light discs were generated using the holographic configuration as shown in FIGS. 9A-C. A 2D pattern of light discs comprising the shape ‘X’ is shown in FIG. 9A, which demonstrates the flexible control over the lateral location of each light disc. The FWHM dimensions of each disc were measured at 1×4×11 μm (in the x, y & z axes respectively). The 3D pattern demonstrates both multiple light discs targeted to the same plane, and also light discs on different planes separated axially by 20 μm, as shown in FIG. 9B. Each light disc was measured to have a FWHM dimensions of 1×4×11 μm in the x, y & z axes respectively. Employing such patterns may be used to simultaneously illuminate numerous cells in different depths, while targeting each cell with multiple light discs. In order to test the targeting capability of the method, two light discs were projected to two separate 10 μm fluorescent beads situated in different planes within a 3D volume as shown in FIG. 9C, left hand side. The result demonstrates an ability to achieve higher cell coverage using light discs in comparison with projection of spots, as shown in FIG. 9C, right hand side.

It is expected that during the life of a patent maturing from this application many relevant optical technologies such as femtosecond lasers, directing and rectification units and optical components for such units and for spatial and for temporal focusing will be developed and the scope of the terms are intended to include all such new technologies a priori.

The terms “comprises”, “comprising”, “includes”, “including”, “having” and their conjugates mean “including but not limited to”.

The term “consisting of” means “including and limited to”.

As used herein, the singular form “a”, “an” and “the” include plural references unless the context clearly dictates otherwise.

It is appreciated that certain features of the invention, which are, for clarity, described in the context of separate embodiments, may also be provided in combination in a single embodiment, and the above description is to be construed as if this combination were explicitly written. Conversely, various features of the invention, which are, for brevity, described in the context of a single embodiment, may also be provided separately or in any suitable subcombination or as suitable in any other described embodiment of the invention, and the above description is to be construed as if these separate embodiments were explicitly written. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. Optical apparatus comprising an impulse producing laser followed in an optical path by a spatio-temporal focusing element configured to provide spectrally dispersed beams, the spatio-temporal focusing element followed in said optical path by a volumetric projection system configured to provide a volumetric image from said collimated beams, the volumetric image extending over a plurality of temporally focused planes.

2. Apparatus according to claim 1, wherein said planes are excitation planes for excitation of fluorescent materials.

3. Apparatus according to claim 1, wherein said impulse producing laser is a femtosecond laser.

4. Apparatus according to claim 1, wherein said spatio-temporal focusing element comprises two dual prism gratings (DPG).

5. Apparatus according to claim 4, further comprising an adjustment mechanism for adjusting a distance between said two DPGs, thereby to adjust a focal spot.

6. Apparatus according to claim 1, wherein said volumetric projection system comprises a scanner in said optical path, the scanner comprising a plurality of galvanometric mirrors to scan spots over a respective focal plane.

7. Apparatus according to claim 1, wherein said volumetric projection system comprises a holographic projection element in said optical path, the holographic projection element configured to provide multiple projections of each spot over a respective focal plane simultaneously.

8. Apparatus according to claim 1, wherein said volumetric projection system comprises a scanner comprising a plurality of galvanometric mirrors to scan each spot over a respective replication plane, and further comprises a holographic projection element configured to provide multiple projections of each spot over a respective focal plane simultaneously.

9. Apparatus according to claim 1, wherein said temporal focusing unit comprises a plurality of mirrors and at least two gratings, configured to provide collimated beams for different wavelengths of said impulse.

10. Apparatus according to claim 1, wherein said temporal focusing unit is modified to elongate said spot into a line.

11. Apparatus according to claim 1, wherein said temporal focusing unit is characterized by an optical axis and is configured for receiving said impulse and for controlling a temporal profile of said impulse to form an intensity peak at said focal plane, said temporal focusing unit having at least two prismatic optical elements configured for receiving said light beam pulse from an input direction parallel to or collinear with said optical axis and diffracting said light beam pulse along said input direction.

12. Apparatus according to claim 1, further comprising a replicator to provide multiple axially shifted optical focal planes.

13. Apparatus according to claim 12, wherein said replicator comprises a plurality of beam splitters and mirrors to provide a plurality of optical paths of different lengths or a diffractive element.

14. Apparatus according to claim 1, used for optogenetic control of a plurality of cells in three dimensions.

15. Apparatus according to claim 1, used for micromachining or microscopic imaging or photo-manipulation or micro-printing.

16. Optical apparatus comprising a temporal focusing unit having two diffraction gratings, a first diffraction grating to separate a laser beam into different wavelengths and a second of said diffraction gratings to recollimate said separated beam, said unit being followed by a volumetric projection system configured to provide a plurality of optical paths for said recollimated beam, thereby to provide a plurality of focal planes in a three-dimensional volume.

17. A method of providing three-dimensional focusing comprising:
providing a femtosecond pulse beam;
providing a first grating to split said femtosecond pulse into a plurality of spectra;
providing a second grating to recollimate said plurality of beams to form a plurality of parallel spectral beams;
splitting said plurality of parallel spectral beams into a plurality of optical paths, thereby to provide a plurality of focal planes in a volume.
